(19) United States
(12) Patent Application Publication
(10) Pub. No.: US 2025/0120836 A1
(43) Pub. Date: Apr. 17, 2025

(54) An apparatus for rehabilitating a joint of a body

(71) Applicants: Aaya Hakeem, Melbourne (AU); Ahmed Hakeem, Melbourne (AU); PHYSIPAL PTY LTD, Melbourne, Victoria (AU)

(72) Inventors: Aaya Hakeem, Melbourne (AU); Jonathan Chapman, Cremorne (AU); Okan Kurt, Istanbul (TR)

(21) Appl. No.: 18/542,815

(22) PCT Filed: Jun. 27, 2022
(86) PCT No.: PCT/AU2022/050656

§ 371 Date: Dec. 18, 2023

(30) Foreign Application Priority Data
Jul. 1, 2021 (AU) 2021204640

(51) Int. Cl. A61F 5/01; A61H 1/02

(52) U.S. Cl. CPC A61F 5/0125; A61H 1/024; A61H 1/0277; A61F 2005/0155; A61F 2005/0167; A61H 2201/018

(57) ABSTRACT

Apparatus for joint range of motion rehabilitation of a wearer of the apparatus is disclosed. The apparatus includes a first support configured to be secured to a first limb portion at a first side of a joint, a second support configured to be secured to a second limb portion at a second side of the joint, and a mechanical system connected to the first support and the second support and operable to control flexion and extension movement of the second support relative to the first support. During the flexion and extension movement, the second support rotates and translates relative to the first support.

### CROSS-REFERENCE TO RELATED APPLICATION

The present application claims priority from Australian Patent Application No. 2021204640 filed on 1 Jul. 2021, the content of which is incorporated herein by reference.

### TECHNICAL FIELD

The present disclosure relates to apparatus and methods for rehabilitating a joint of a body.

### BACKGROUND

Joint Range of Motion (ROM) rehabilitation is typically carried out following knee arthroplasty, such as partial or full knee replacement, in an attempt to restore normal range of motion to a desirable extent. Certain pathologies of the knee may also require range of motion rehabilitation.

Other joints of the human body, such as the elbow, ankle, wrist, hip, and shoulder joints, etc., can also require joint range of motion rehabilitation following similar surgical procedures or due to certain pathologies.

While patients can perform their own ROM rehabilitation at home, regular visits from or to a physiotherapist is typically required, particularly when the patient is weaker or when more complex exercises are to be performed.

Time lost between visits from or to a physiotherapist to perform RoM exercises can significantly delay the time taken to restore the desired range of motion of a joint.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to one aspect of the present disclosure, there is provided an apparatus for joint range of motion rehabilitation of a wearer of the apparatus, the apparatus comprising:
a first support configured to be secured to a first limb portion at a first side of a joint;
a second support configured to be secured to a second limb portion at a second side of the joint; and
a mechanical system connected to the first support and the second support and operable to control flexion and extension movement of the second support relative to the first support, wherein during the flexion and extension movement, the second support rotates and translates relative to the first support.

In some embodiments, the joint may be a knee joint, elbow joint or ankle joint, for example. Thus, the first and second limb portions may be upper and lower leg portions (knee joint), upper and lower arm portions (elbow joint) or lower leg and foot portions (ankle joint). Nevertheless, the apparatus may be used with a variety of other joints and limb portions such as hip, shoulder, wrist, finger joints or otherwise. In some embodiments, the apparatus may be generally configured as a brace, e.g. a knee brace, elbow brace or ankle brace, etc.

In some embodiments, the mechanical system may comprise an actuator configured to partially or entirely drive the flexion and extension movement of the second support relative to the first support. In some embodiments, the actuator may comprise a handle.

The actuator may be operable by the wearer of the apparatus. Additionally or alternatively, the actuator may be operable by a physiotherapist or other caregiver. In some embodiments, the actuator may be removable from the apparatus, e.g. to reduce the size of the apparatus for storage or otherwise. In some embodiments, the mechanical system may be entirely manually operable, although in other embodiments the actuator of the mechanical system may comprise a powered drive means such as a motor to partially or entirely drive the flexion and extension movement. Manual operation of the mechanical system may in some instances include the wearer partially or entirely driving the flexion and extension movement of the second support relative to the first support by carrying out native flexion and extension movement of the second limb portion relative to the first limb portion.

The arrangement of the apparatus may make it particularly suitable for self-administrable therapy, independent of a physiotherapist, and thus a desired ROM of a joint may be achieved in a significantly reduced time period and in a more efficient manner.

In some embodiments, the mechanical system may comprise a mechanism configured to control or enable the rotation and translation of the second support relative to the first support during the flexion and extension movement. The mechanical system may comprise a housing and the mechanism may be at least partly located in the housing.

In some embodiments, the mechanism may comprise a gear system and/or mechanical linkage, for example. The gear system may be a planetary gear system, for example. In some embodiments, to achieve both the rotational and translational movement, the mechanism may comprise a cam member or cam surface and/or may comprise asymmetrically configured gearing or mechanical linkage components. For example, a twin asymmetric planetary gear system may be utilised in some embodiments.

In some embodiments, the mechanical system may comprise first and second arms configured to be fixed to the first support and the second support, respectively. The arms may be elongate and substantially rigid (e.g. by being formed of rigid plastic or metal) and configured to extend partially or entirely along a length of the respective support. The first and second arms may rotate with the first and second supports, respectively, and generally assist in the transfer of forces between the mechanical system and the respective supports. In some embodiments, the first and second arms may rotate by being connected via a gear system as described above, or the first and second arms may rotate by being connected via a hinge, for example.

In some embodiments, at least one of the first and second arms may be adjustable in length. For example, at least one of the first and second arms may be adjustable in length by comprising first and second arm portions that are slidable relative to each other, e.g. by being configured in a telescoping arrangement or otherwise. The first arm portion may be fixed to a respective portion of the hinge and the second arm portion may be fixed to the respective support. Therefore, while the first and second supports rotate relative to each other by virtue of the hinge, the first and second supports can also translate relative to each other upon adjusting of the length of the second arm. During the flexion and extension movement of the second support relative to the first support, and therefore the rotation of the second arm relative to the first arm, an effective length of the at least one of the first and second arms having the adjustable length, e.g. the second arm fixed to the second support, may adjust to enable the translation of the second support relative to the first support. The adjustable arm may enable the translation of the second support relative to the first support as an addition to, or as an alternative to, use of other mechanism features such as the gear system and/or mechanical linkage described above.

The arrangement of the different embodiments of the mechanism may ensure that the first and second supports do not rotate about a uniform centre of rotation. For example, in some embodiments, a pivot point between the first and second supports may move along an arcuate path during the flexion and extension movement.

In some embodiments, the housing may be configured to be positioned substantially adjacent a centre of rotation of the joint when the first support and the second support are secured to the first limb portion and the second limb portion, respectively. In some embodiments, the actuator may form part of, or be configured to engage with, the housing.

In some embodiments, the translation of the second support relative to the first support may be configured to cause or enable an anterior-posterior translation of the second limb portion relative to the first limb portion. For example, the translation of the second support relative to the first support may be configured to cause or enable an anterior translation of the second limb portion relative to the first limb portion during extension movement of the second support relative to the first support and a posterior translation of the second limb portion relative to the first limb portion during flexion movement of the second support relative to the first support.

In addition to or as an alternative to the anterior-posterior translation, the translation of the second support relative to the first support may be configured to cause or enable an axial translation of the second limb portion relative to the first limb portion.

In some embodiments, the flexion and extension movement may comprise at least one phase wherein the rotation and the translation of the second support relative to the first support occurs simultaneously. In some embodiments, for example, the entire flexion and extension movement, between maximum degrees of flexion and extension, may comprise simultaneous rotation and translation of the second support relative to the first support. Alternatively, the flexion and extension movement may comprise at least one phase wherein the rotation of the second support relative to the first support occurs substantially without the translation of the second support relative to the first support.

The rate of translation of the second support relative to the first support may be non-uniform during the flexion and extension movement. For example, in some embodiments, during a full flexion movement of the second support relative to the first support, from a maximum degree of extension to a maximum degree of flexion, a highest rate of translation of the second support relative to the first support may occur during an intermediate phase of the full flexion movement. The intermediate phase may be between 20% and 60%, or 30% and 50%, of the full flexion movement, for example.

In some embodiments, during a full flexion movement of the second support relative to the first support, from a maximum degree of extension to a maximum degree of flexion, a lowest rate of translation, or substantially no translation, of the second support relative to the first support, may occur during a beginning and/or end phase of the full flexion movement. The beginning phase may be between 0% and 30%, or 0% and 20%, of the full flexion movement, for example. The end phase may be between 50% and 100%, or 60% and 100%, of the full flexion movement, for example.

In the above examples, while the rotation and translation of the second support relative to the first support during a full flexion movement is described, it will be recognised that the rotation and translation of the second support relative to the first support can occur in reverse for a full extension movement. In this regard, the first and second supports may move backwards and forwards along the same path during the flexion and extension movements, respectively. Nevertheless, it is conceived that the mechanical system may alternatively be configured so that the first and second support may move backwards and forwards along different paths during the flexion and extension movements, respectively.

By configuring the mechanical system so that, during the flexion and extension movements, the first support and the second support both rotate and translate relative to one another, the apparatus may provide for enhanced ROM rehabilitation. For example, the movement of the first and second supports of the present mechanical system may provide for a more natural or native movement of the first and second limb portions either side of the joint. Additionally or alternatively, the movement of the first and second supports may provide a more effective rehabilitation movement of the first and second limb portions.

In some embodiments, the apparatus may comprise one or more extension and/or flexion limiters configured to limit a maximum degree of extension and/or flexion of the second support relative to the first support. The extension and/or flexion limiter may ensure that the wearer does not over-extend, e.g. hyperextend, and/or over-flex, e.g. hyperflex, the limb portions when using the apparatus. In some embodiments, the extension and/or flexion limiter may be adjustable to change the maximum degree of extension and/or flexion of the second support relative to the first support. For example, the extension and/or flexion limiter may be a movable blocking element having an abutment surface that abuts one or more components of the mechanical system and the mechanism that controls or enables the rotation and translation of the second support relative to the first support, when a desired maximum degree of extension and/or flexion is reached.

In some embodiments, the apparatus may comprise one or more indicators configured to indicate a degree of flexion of the second support relative to the first support achieved during the flexion movement and/or a degree of extension of the second support relative to the first support achieved during the extension movement. In some embodiments, the indicators may indicate a maximum degree of flexion of the second support relative to the first support achieved during the flexion movement and/or a maximum degree of extension of the second support relative to the first support achieved during the extension movement. Each indicator may be in the form of a dial. Each indicator may comprise a selection element, and one or more indicia elements movable relative to the selection element during the flexion and/or extension movement. The selection element may be a window, line or pointer, for example. The one or more indicia elements may be a colour or shading gradient, a plurality of different numbers (e.g. indicating specific numerical degrees or angles of rotation), or symbols, for example. In this regard, any reference in the present specification to indication of the “degree” of flexion and/or extension need not necessarily require indication of a specific numerical degree (or angle) of rotation, but may include a more general indication of an extent to which flexion or extension has occurred, or is occurring. The alignment of a particular indicia element or portion of indicia element with the selection element may be indicative of the degree of flexion and/or extension movement at a particular point in time. In some embodiments, to indicate the maximum degree of flexion and/or extension achieved, an indicia element or portion of indicia element may be configured to remain aligned with the selection element until the maximum degree of flexion and/or extension is subsequently exceeded.

In some embodiments, the one or more indicators may be positioned to be directly observable by (e.g., in line of sight of) the wearer of the apparatus. For example, the one or more indicators may be positioned on or at a peripheral surface of the housing. The housing may be of a cylindrical or drum shape, for example, and the one or more indicators may be positioned on or at a curved peripheral wall of the housing. The peripheral surface or curved peripheral wall may be configured to face in a proximal or anterior direction or the limb on which the apparatus is worn, or a generally superior or anterior direction of the wearer's body.

In some embodiments, one or both of the first support and the second support may comprise one or more straps to be secured to the respective first limb portion or second limb portion. Additionally or alternatively, one or both of the first support and the second support may comprise one or more sleeve portions configured to be secured to the respective first limb portion or second limb portion. The sleeve portions may be elasticated for example, and may comprise elasticated rubber such as neoprene (polychloroprene), for example. In some embodiments, one or both of the first support and the second support may be secured to the respective first limb portion or second limb portion using one or more fasteners such as a hook and loop fastener; a buckle fastener; a snap fastener; and/or a hook-eye fastener. The fastener may be partially or entirely comprised in the straps, the sleeve portions or other components of the supports. In some embodiments, the first support and the second support may comprise one or more pads to ensure comfort for the wearer.

When the first and/or second supports comprise sleeve portions, the sleeve portions may have a rigid or semi-rigid section receiving section that is fixed to the respective first and second arm of the mechanical system. The receiving section may be fixed to the sleeve portion via an adhesive, for example, and the arm may be fixed to the receiving section using adhesive and/or screws for example.

According to another aspect of the present disclosure, there is provided a method of performing joint range of motion rehabilitation comprising:
securing a first support to a first limb portion of a wearer at a first side of a joint;
securing a second support to a second limb portion of the wearer at a second side of the joint; and
operating a mechanical system connected to the first support and the second support to control flexion and extension movement of the second support relative to the first support, wherein during the flexion and extension movement, the second support rotates and translates relative to the first support.

In some embodiments, operation of the mechanical system may comprise use of an actuator to partially or entirely drive the flexion and extension movement of the second support relative to the first support. However, in other embodiments, operation of the mechanical system may comprise the wearer partially or entirely driving the flexion and extension movement of the second support relative to the first support by carrying out native flexion and extension movement of the second limb portion relative to the first limb portion.

In the present disclosure, while focus is given to apparatus having a mechanical system configured so that a first support and a second support rotate and translate relative to one another, it will be recognised that a variety of features disclosed herein may still be used advantageously without the first support and the second support necessarily rotating and translating relative to one another. For example, the configuration of the supports, the flexion or extension limiters, the housing, the arms, and/or indicators, etc., may be used advantageously within apparatus in which the first support and the second support rotate relative to one another but do not necessarily translate relative to one another.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments are now described with reference to the accompanying drawings, in which:

FIG. 1 shows a perspective, partly exploded view of an apparatus according to an embodiment of the disclosure;

FIG. 2 shows a perspective view of the apparatus according to FIG. 1 with an actuator and a housing wall removed;

FIG. 3 shows an exploded view of a mechanical system of the apparatus of FIG. 1;

FIGS. 4A to 4C show front views of the mechanical system of the apparatus of FIG. 1 at different states of flexion and extension;

FIGS. 5A to 5C show rear views of the mechanical system of the apparatus of FIG. 1 at different states of flexion and extension;

FIG. 6 shows an exploded view of a mechanical system according to another embodiment of the present disclosure;

FIGS. 7A to 7C show front views of a mechanical system of FIG. 6 at different states of flexion and extension;

FIGS. 8A to 8D show front views of a mechanical system according to another embodiment of the present disclosure at different states of flexion and extension;

FIG. 9 shows a perspective view of a mechanical system according to another embodiment of the present disclosure; and

FIGS. 10A and 10B show perspective, partly exploded, views of an apparatus according to another embodiment of the present disclosure.

### DESCRIPTION OF EMBODIMENTS

One or more embodiments of the present disclosure may provide, at least in part, an apparatus for joint range of motion rehabilitation.

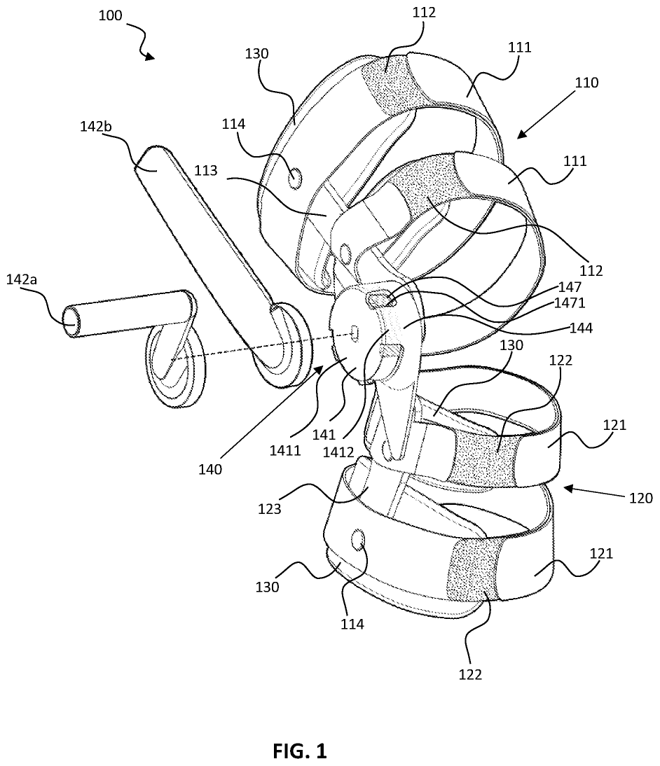

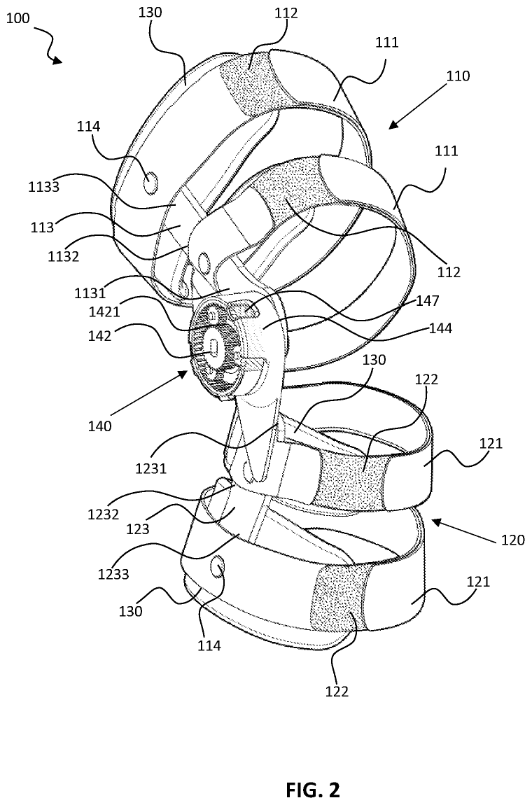

FIGS. 1 and 2 show an apparatus 100 for joint range of motion rehabilitation according to an embodiment of the present disclosure, the apparatus comprising: a first support 110 configured to be secured to a first limb portion at a first side of a joint and a second support 120 configured to be secured to a second limb portion at a second side of the joint (the joint and limb portions not being illustrated in the Figures). The apparatus 100 includes a mechanical system 140 connected to the first support 110 and the second support 120 and which is operable to control flexion and extension movement of the second support 120 relative to the first support 110 (and thus the second limb portion relative to the first limb portion). The mechanical system 140 is configured so that, during the flexion and extension movement, the second support 120 rotates and translates relative to the first support 110.

In this embodiment, the apparatus 100 is in the form of a knee brace, and configured for rehabilitation of a knee. Thus, the first and second limb portions are the upper leg and lower leg portions, respectively, that are located either side of the knee. Nevertheless, the apparatus 100 may be configured for use with a variety of other joints and limb portions such as elbow, ankle or finger joints, or otherwise.

In this embodiment, the first support 110 and the second support 120 of the apparatus 100 each include attachment devices, in the form of straps 111, 121, that are spaced apart from each other and configured to extend around the limb portions to secure the supports 110, 120 to the limb portions. As illustrated in FIG. 1, both the first support 110 and the second support 120 comprise two straps 111, 121 each, although any number of straps 111, 121 may be provided in other embodiments. The straps 111, 121 are held in a looped configuration around the respective limb portions formed using fasteners 112, 122. In this embodiment, the fasteners are hook and loop elements (e.g. Velcro™), although alternative or additional fasteners can be utilised, such as buttons, buckles, belts, snaps, etc. The straps 111, 121 may be adjustably fastened, e.g. with differing degree of tightness around the respective limb portions. Moreover, the apparatus 100 including the supports 111, 121 may be provided in different sizes (i.e. XS, S, M, L, XL) to suit wearers of different sizes.

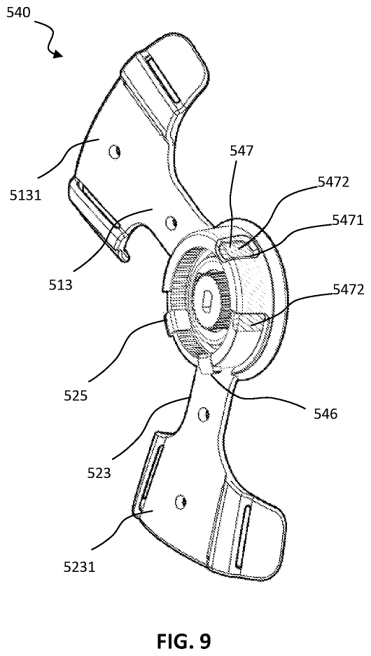

To assist in transferring forces between the mechanical system 140 and the first and second supports 110, 120, the mechanical system 140 includes first and second arms 113, 123, each arm 113, 123 being generally elongate and substantially rigid. Each arm 113, 123 is fixed to portions of the supports 110, 120, such as the straps 111, 112. In this embodiment, with reference to FIG. 2, a proximal end region 1131, 1231 of each arm 113, 123 is engaged with a mechanism 142 of the mechanical system 140, and an intermediate end region 1132, 1232 and distal end region 1133, 1233 of each arm 113, 123 is fixed to a respective strap 111, 121. Fixing of each region of the arm 113, 123 to the respective strap 111, 121 is through use of a rivet, screw and/or other fixing element 114. Moreover, the arms 113, 123 extend under or through portions of the straps 111, 121. In some embodiments, the arms 113, 123 may have one or more laterally extending portions to spread forces at the point of fixing to the respective strap 111, 121 (see e.g. discussions of FIG. 9 below). Moreover, one or more pads 130 may be provided under the point of fixing to improve comfort for the wearer.

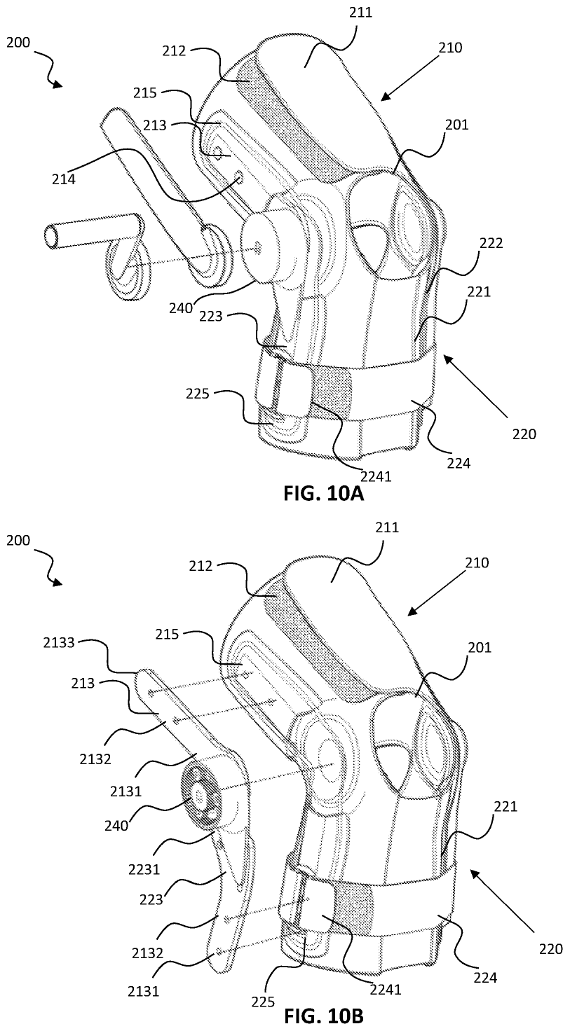

In an alternative embodiment, with reference to FIGS. 10A and 10B, an apparatus 200 for joint range of motion rehabilitation is provided that is similar to the apparatus 100 described above, except for the configuration of its supports 210, 220. In particular, rather than the supports having straps that are spaced apart from each other, each support 210, 220 in this embodiment includes a respective sleeve portion 211, 221. In this embodiment, the sleeve portions 211, 221 are parts of a continuous sleeve that is configured to extend across the joint in use, the continuous sleeve including an optional opening 201 to receive a protruding anterior portion of the knee. However, in alternative embodiments, discrete sleeve portions, e.g. separate individual sleeves, may be provided. One or both of the sleeve portions 211, 221 in this embodiment are formed of a flap, arranged to be held together in a sleeve configuration around the respective limb portions and formed using one or more fasteners 212. In this embodiment, the fasteners 212 are hook and loop fasteners (e.g. Velcro™) although alternative or additional fastening means can be utilised, such as buttons, buckles, belts, snaps, etc. The sleeve portions 211, 221 may be adjustably fastened, e.g. with differing degree of tightness around the respective limb portions. Moreover, the apparatus 200 including the supports may be provided in different sizes (i.e. XS, S, M, L. XL) to suit wearers of different sizes. In this embodiment an additional fastening strap 224 is provided to further secure the second support 220 to the respective limb portion, the strap 224 including a buckle and hook and loop fastener 2241.

Again, to assist in transferring forces between the mechanical system 240 and the first and second supports 210, 220, the mechanical system 240 includes first and second arms 213, 223, each arm 213, 223 being elongate and substantially rigid. Each arm 213, 223 is fixed to portions of the supports 210, 220, and in particular the sleeve portions 211, 221. The supports 210, 220 each have a rigid or semi-rigid receiving section 215, 225 that is fixed to the respective sleeve portion 211, 221 via an adhesive, for example, and the arm 213, 223 is received by and fixed to the receiving section 215, 225 using adhesive and/or screws or rivets 214, for example. In this embodiment, a proximal end region 2131, 2231 of each arm 213, 223 is engaged with a mechanism of the mechanical system 240, and an intermediate end region 2132, 2232 and distal end region 2133, 2233 of each arm 213, 223 is fixed to the receiving section 215, 225.

In any embodiment, the first and second supports 110, 120, 210, 220 including the straps or sleeve portions, can be fabricated from any suitable materials. The materials may be water resistant, breathable and/or at least partially elastic, for example. The materials may include an antibacterial material, to neutralize odour, a padding material, such as foam, to increase comfort and durability, and/or a gripping material to increase the grip of the apparatus against the first limb portion and the second limb portion to avoid migration of the apparatus during the flexion and extension movement.

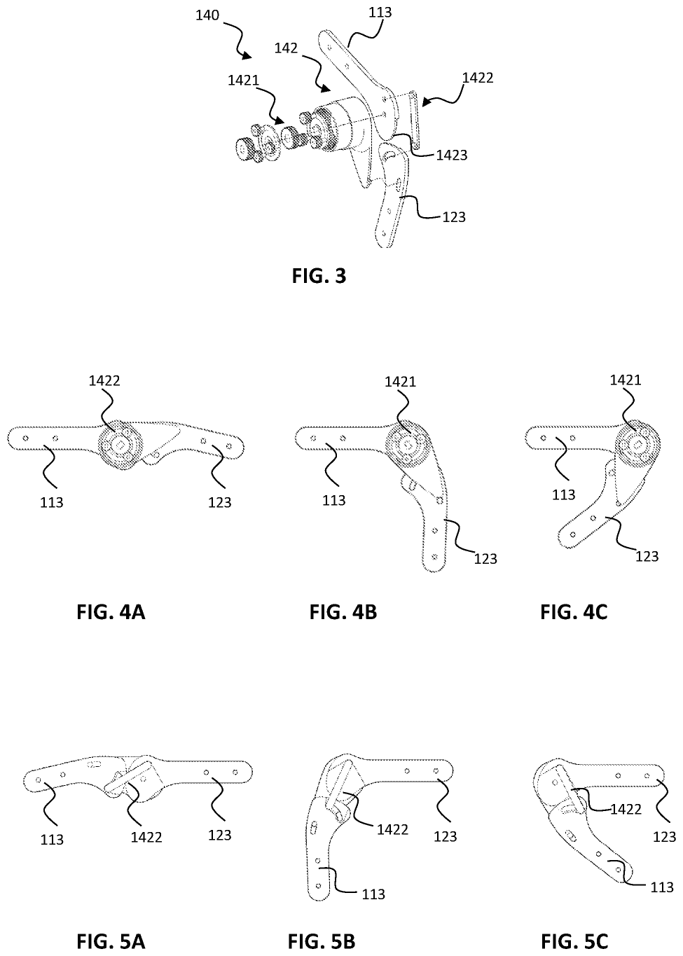

The features and operation of the mechanical system 140 are now described in more detail with reference to the embodiment illustrated in FIGS. 1 and 2, and with further reference to FIGS. 3 to 5C. The mechanical system 140 includes an actuator 142a, 142b configured to partially or entirely drive the flexion and extension movement of the second support 120 relative to the first support 110. The actuator may be or may include a handle 142a, 142b. The handle 142a, 142b may be in a variety of forms, such as a crank handle 142a, a lever handle 142b, or otherwise. In the Figures, both a crank handle 142a and a lever handle 142b are illustrated, although typically only one of these handles would be included in the apparatus 100.

Advantageously, the handle 142a, 142b may be operable by the wearer of the apparatus 100. Additionally or alternatively, however, the handle 142a, 142b may be operable by a physiotherapist or other caregiver. In some embodiments, the handle 142a, 142b may be removable from the apparatus 100, e.g. to reduce the size of the apparatus 100 for storage, to enable use of the apparatus like a traditional brace (e.g. under clothing), or otherwise.

The mechanical system 140 of the present embodiment is manually operable, although in other embodiments the actuator of the mechanical system may comprise a powered drive means such as a motor to partially or entirely drive the flexion and extension movement. In the present embodiment, in addition to or instead of using the handle 142a, 142b, manual operation of the mechanical system may include the wearer partially or entirely driving the flexion and extension movement of the second support 120 relative to the first support 110 by carrying out native flexion and extension movement of the second limb portion relative to the first limb portion.

The arrangement of the apparatus 100 may make it particularly suitable to allow for self-administrable therapy, independent of a physiotherapist, and thus a desired range of motion of a joint may be achieved in a significantly reduced time period and in a more efficient manner.

In this embodiment, the mechanical system 140 includes a housing 141 and a mechanism 142 (see FIG. 2) to control or enable the rotation and translation of the second support 120 relative to the first support 110, the mechanism 142 being partly located in the housing 141. The housing 141 is configured to be positioned substantially adjacent a centre of rotation of the joint when the first support 110 and the second support 120 are secured to the first limb portion and the second limb portion, respectively.

The housing 141 is a substantially cylindrical or drum shaped housing in this embodiment, including a lateral wall 1411, which is substantially circular and flat in this embodiment, and a peripheral/side wall 1412, which is curved and extends around a peripheral edge of the lateral wall 1411 in this embodiment. The handle 142a, 142b engages the housing 141 via an opening in the lateral wall 1411.

With reference to FIGS. 2 and 3, the mechanism 142 in this embodiment includes a gear system 1421 and a mechanical linkage 1422. The mechanism 142 is configured to provide both the desired rotation and translational movement of the supports 110, 120, and optionally also to provide mechanical advantage to assist the user in manually operating the apparatus. In this embodiment, the gear system 1421 is a planetary gear system (see e.g. FIG. 3 and FIGS. 4A to 4C), which works in conjunction with the mechanical linkage 1422 to control movement of the arms 113, 123 of the mechanical system 140. The mechanical linkage 1421 is arranged to convert a rotation of the planetary gear system 1421, about a uniform centre of rotation, to both a rotational and translational movement of the first and second arms 113, 123 and therefore supports 110, 120. To achieve this in part, the mechanical linkage 1422 includes pins engageable in, arcuate slots and a cam member or cam surface 1423.

FIGS. 4A to 4C, and 5A to 5C, illustrate how the arms 113, 123, rotate and translate relative to each other as the mechanism 142 is actuated, from a maximum degree of extension (FIGS. 4A and 5A) through an intermediate state of extension/flexion (FIGS. 4B and 5B) to a maximum degree of flexion (FIGS. 4C and 5C). In this embodiment, the mechanism 142 is configured such that a pivot point between the arms 113, 123 moves along an arcuate path during the flexion and extension movement.

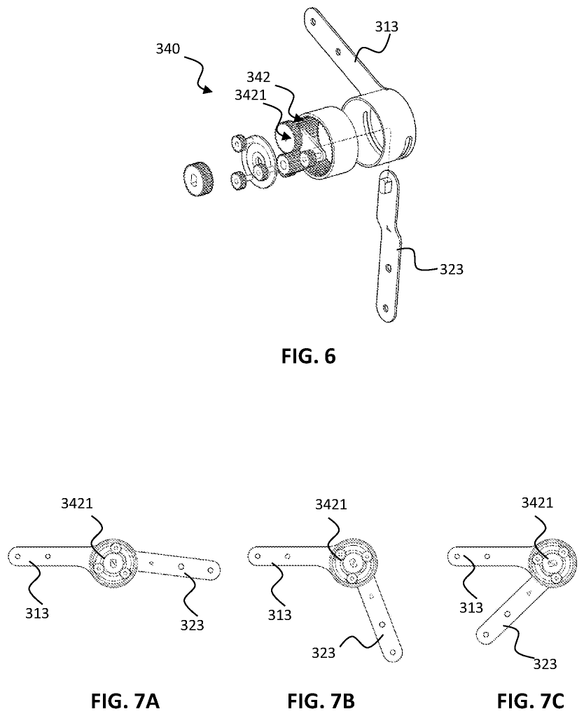

A mechanism 342 of a mechanical system 340 according to an alternative embodiment of the present disclosure is illustrated in FIGS. 6 to 7C. In this embodiment, the mechanism 342 includes an asymmetrically configured planetary gear system 3421, and particularly a twin asymmetric planetary gear system, to provide both the desired rotation and translational movement of arms 313, 323 and therefore supports (not illustrated) that are fixed to the arms 313, 323, and optionally also to provide mechanical advantage to assist the user in manually operating the apparatus.

FIGS. 7A to 7C illustrate how the arms 313, 323, rotate and translate relative to each other as the mechanism 342 is actuated, from a maximum degree of extension (FIG. 7A) through an intermediate state of extension/flexion (FIG. 7B) to a maximum degree of flexion (FIG. 7C). Again, in this embodiment, the mechanism 342 is configured such that a pivot point between the arms 313, 323 moves along an arcuate path during the flexion and extension movement.

In the above-described embodiments, the mechanical system 140, 240, 340 is configured such that translation of the second support 120, 220 relative to the first support 110, 210 causes or enables an anterior-posterior translation of the second limb portion relative to the first limb portion. In particular, the translation of the second support 120, 220 relative to the first support 110, 210 causes or enables an anterior translation of the second limb portion relative to the first limb portion during extension movement of the second support 120, 220 relative to the first support 110, 210 and a posterior translation of the second limb portion relative to the first limb portion during flexion movement of the second support 120, 220 relative to the first support 110, 210. Additionally, the translation of the second support 120, 220 relative to the first support 110, 210 causes or enables a degree of axial translation of the second limb portion relative to the first limb portion.

The mechanical system 140, 240, 340 is configured so that the flexion and extension movement comprises one or more phases wherein the rotation and the translation of the second support 120, 220 relative to the first support 110, 210 occurs simultaneously, and optionally, one or more phases where rotation of the second support 120, 220 relative to the first support 110, 210 occurs substantially without the translation of the second support 120, 220 relative to the first support 110, 210.

The mechanical system 140, 240, 340 is configured so that the rate of translation, and particularly the rate of anterior-posterior translation, of the second support 120, 220 relative to the first support 110, 210, is non-uniform during the flexion and extension movement. During a full flexion movement of the second support 120, 220 relative to the first support 110, 210 from a maximum degree of extension to a maximum degree of flexion, a highest rate of translation of the second support 120, 220 relative to the first support 110, 210 occurs during an intermediate phase of the full flexion movement. The intermediate phase is between 20% and 60%, or 30% and 50%, of the full flexion movement, in this embodiment.

During a full flexion movement of the second support 120, 220 relative to the first support 110, 210 from a maximum degree of extension to a maximum degree of flexion, a lowest rate of translation, particularly anterior-posterior translation, or substantially no translation, of the second support 120, 220 relative to the first support 110, 210, occurs during the beginning and end phases of the full flexion movement. The beginning phase is between 0% and 30%, or 0% and 20%, of the full flexion movement, in this embodiment. The end phase is between 50% and 100%, or 60% and 100%, of the full flexion movement, in this embodiment.

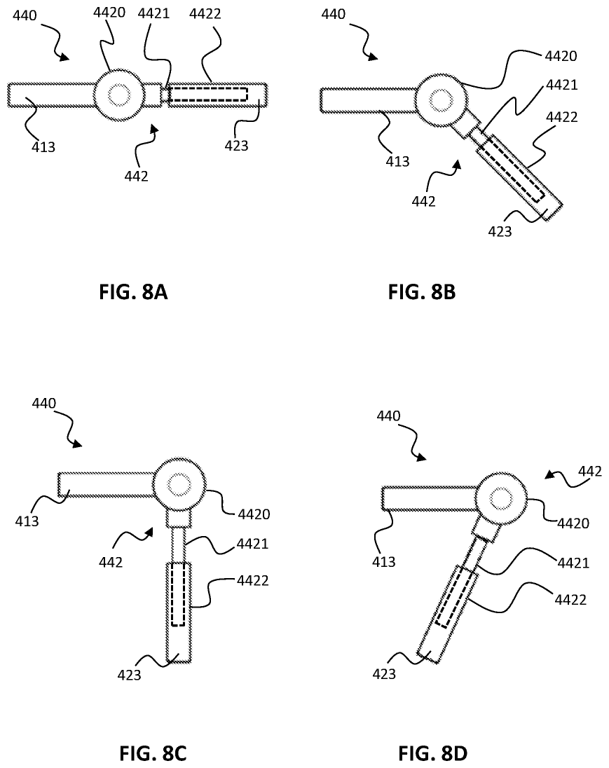

A mechanical system 440 according to an alternative embodiment of the present disclosure is illustrated in FIGS. 8A to 8D. In this embodiment, the mechanical system 440 comprises first and second arms 413, 423 and a mechanism 442 comprising a hinge 4420, the first and second arms 413, 423 being connected to respective portions of the hinge 4420 such that the first and second arms 413, 423 rotate relative to each other during the flexion and extension movement of the second support relative to the first support. The first and second arms 413, 423 are configured to be fixed to portions of the first and second supports, respectively, e.g. in accordance with the connection arrangements discussed in the previous embodiments.

In this embodiment, the hinge 4420 is configured to be positioned substantially adjacent a centre of rotation of the joint when the first and second supports are secured to the first and second limb portions, respectively. The hinge 4420 may be configured to receive or connect to an actuator (not illustrated), such as a handle, the actuator configured to partially or entirely drive the flexion and extension movement of the second support relative to the first support.

FIGS. 8A to 8D illustrate the mechanism 442 being actuated from a maximum degree of extension (FIG. 8A) through intermediate states of extension/flexion (FIGS. 8B and 8C) to a maximum degree of flexion (FIG. 8D). In this embodiment, the first and second arms 413, 423 are configured such that they rotate about a uniform centre of rotation during the flexion and extension movement of the second support relative to the first support, the uniform centre of rotation being generally defined by the hinge 4420.

At least one of the first and second arms 413, 423, and particularly the second arm 423 in this embodiment, is adjustable in length. The second arm 423 is adjustable in length by comprising first and second arm portions 4421, 4422 that are slidable relative to each other, e.g. by being configured in a telescoping arrangement. The first arm portion 4421 is fixed to the respective portion of the hinge 4420 and second arm portion 4422 is fixed to the second support. Therefore, while the first and second supports relative to each other by virtue of the hinge 4420, the first and second supports can also translate relative to each other upon adjusting of the length of the second arm 423. In general, the hinge 4420 and first and second arm portions 4421, 4422 of the second arm 423 of the mechanical system 440 are configured to work in conjunction to enable both the desired relative rotational and translational movements of first and second supports, and optionally to provide mechanical advantage to assist the user in manually operating the apparatus.

In this embodiment, while the first and second arms 413, 423 rotate about a uniform centre or rotation during the flexion and extension movement of the second support relative to the first support, the second support can be translated relative to the first support due to the adjusting of the length of the second arm 423 and such that such that a notional pivot point between the first and second supports moves along an arcuate path during the flexion and extension movement.

In this embodiment, the mechanical system 440 is configured such that, during the flexion and extension movement and therefore during the rotation of the second arm 423 relative to the first arm 413, translation of the second support relative to the first support is achieved through adjustment of the length of the second arm 423, thereby causing or enabling relative movement of respective limb portions that are secured to the first and second supports. The relative rotational and translational movements of the limb portions, including the rate of change of these movements, may be similar or identical to that described above with respect to previous embodiments.

In an alternative embodiment, the length adjustable arm as described above may be employed in conjunction with other mechanism features such as the gear system and/or mechanical linkage described in embodiments above, in order to assist in the translation of the second support relative to the first support.

In the above description of the mechanical system 140, 240, 340, 440, while the translation during a full flexion movement is described, the arrangement is substantially reversed for a full extension movement. In this regard, the first and second supports 110, 210, 120, 220 can move backwards and forwards along the same path during the flexion and extension movements, respectively. Nevertheless, it is conceived that the mechanical system 140, 240, 340, 440 may alternatively be configured so that the first and second supports 110, 210, 120, 220 may move backwards and forwards along different paths during the flexion and extension movements, respectively. In the above description, the specific nature of the rotation and translation may be particularly suited to rehabilitation of a knee joint, and may be adapted for other joints if necessary.

By configuring the mechanical system 140, 240, 340, 440 so that, during the flexion and extension movement, the first support 110, 210 and the second support 120, 220 both rotate and translate relative to one another (e.g., rather than rotate only), the apparatus 100, 200 provides for enhanced joint range of motion rehabilitation. For example, the movement of the first and second supports 110, 210, 120, 220 of the mechanical system 140, 240, 340, 440 provides for a more natural or native movement of the first and second limb portions either side of the joint.

In some embodiments, the apparatus 100, 200 includes one or more flexion and/or extension limiters configured to limit a maximum degree of extension and/or flexion of the second support 120, 220 relative to the first support 110, 210. A portion of a mechanical system 540, similar to those discussed above, but which includes a flexion limiter 545 and an extension limiter 546 is illustrated in FIG. 9. The extension limiter 546 ensures that the wearer does not over-extend, e.g. hyperextend, the limb portions, and the flexion limiter 545 ensures that the wearer does not over-flex, e.g. hyperflex, the limb portions, when using apparatus according to the present disclosure. Both the flexion limiter 545 and the extension limiter 546 are adjustable in this embodiment to change the maximum degree of extension and flexion of the second support 120, 220 relative to the first support 110, 210. Each limiter 545, 546 is in the form of a movable blocking element having an abutment surface that abuts one or more other components (not illustrated) of the mechanical system 540 when the desired maximum degree of extension or flexion is reached in order to restrict any further extension or flexion movement. As seen in FIG. 9, arms 513, 523 of the mechanical system 540 each include laterally extending portions 5131, 5231 to spread forces at the point of fixing to supports.

In some embodiments, the apparatus 100, 200 includes at least one indicator 147, 547 configured to indicate a degree of flexion of the second support 120, 220 relative to the first support 110, 210 achieved during the flexion movement and/or a degree of extension of the second support 120, 220 relative to the first support 110, 210 achieved during the extension movement. An indicator 147 is included in the mechanical system 140 illustrated in FIGS. 1 and 2, and an indicator 547 is also included in the partial mechanical system 540 illustrated in FIG. 9.

The indicator 147, 547 may be generally in the form of a dial, and may include a selection element, which can be in the form of a window 5471, and one or more indicia elements 5472, which are movable relative to the selection element 5471 during the flexion and/or extension movement. An indicia element 5472 in this embodiment is provided by a colour or shading gradient. However, in alternative embodiments a sequences of numbers (e.g. numbers indicating a specific numerical value of angle and/or degree of rotation) or symbols representative of degrees of rotation may be used, which numbers or symbols sequentially align with the window 5471 during the flexion and extension movement. The alignment of the one or more indicia elements, or portions thereof, with the window 5471, is indicative of the degree of flexion and/or extension at a particular point in time. In some embodiments, to indicate the maximum degree of flexion and/or extension achieved, the indicia element or a portion of the indicia element may be configured to remain aligned with the selection element, until the maximum degree of flexion and/or extension is subsequently exceeded.

In these embodiments, the indicator 147, 547 is positioned so that it can be directly observed by the wearer of the apparatus. As seen in FIGS. 1 and 2, for example, the indicator 147 is positioned at the peripheral wall 1412 of the housing (a window 1471 of the indicator 147 being located in the peripheral wall 1412) such that the indicator 147 faces in a proximal and/or anterior direction of the limb on which the apparatus is worn, or a generally superior or anterior direction of the wearer's body.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. Apparatus for joint range of motion rehabilitation of a wearer of the apparatus, the apparatus comprising:
a first support configured to be secured to a first limb portion at a first side of a joint;
a second support configured to be secured to a second limb portion at a second side of the joint; and
a mechanical system connected to the first support and the second support and operable to control flexion and extension movement of the second support relative to the first support, wherein during the flexion and extension movement, the second support rotates and translates relative to the first support.

2. The apparatus of claim 1, wherein the mechanical system comprises an actuator configured to partially or entirely drive the flexion and extension movement of the second support relative to the first support.

3. The apparatus of claim 2, wherein the actuator comprises a handle.

4. The apparatus of claim 2 or 3, wherein the actuator is operable by the wearer of the apparatus.

5. The apparatus of any one of claims 2 to 4, wherein the actuator is removable from the apparatus.

6. The apparatus of any one of claims 2 to 5, wherein the actuator comprises a motor.

7. The apparatus of any one of the preceding claims, wherein the mechanical system comprises a mechanism to control or enable the rotation and translation of the second support relative to the first support.

8. The apparatus of claim 7, wherein the mechanical system comprises a housing, the mechanism at least partly located in the housing.

9. The apparatus of claim 7 or claim 8, wherein the mechanism comprises a mechanical linkage.

10. The apparatus of any one of claims 7 to 9, wherein the mechanism comprises a gear system.

11. The apparatus of claim 10, wherein the gear system is a planetary gear system.

12. The apparatus of any one of claims 9 to 11, wherein the housing is configured to be positioned substantially adjacent a centre of rotation of the joint when the first support and the second support are secured to the first limb portion and the second limb portion, respectively.

13. The apparatus of claim 7, wherein the mechanism comprises a hinge configured to be positioned substantially adjacent a centre of rotation of the joint when the first support and the second support are secured to the first limb portion and the second limb portion, respectively.

14. The apparatus of any one of the preceding claims, wherein the mechanical system is configured such that a pivot point between the first and second supports moves along an arcuate path during the flexion and extension movement.

15. The apparatus of any one of the preceding claims, wherein the translation of the second support relative to the first support is configured to cause an anterior-posterior translation of the second limb portion relative to the first limb portion.

16. The apparatus of claim 15, wherein the translation of the second support relative to the first support is configured to cause an anterior translation of the second limb portion relative to the first limb portion during extension movement of the second support relative to the first support and a posterior translation of the second limb portion relative to the first limb portion during flexion movement of the second support relative to the first support.

17. The apparatus of any one of the preceding claims, wherein the translation of the second support relative to the first support is configured to cause an axial translation of the second limb portion relative to the first limb portion.

18. The apparatus of any one of the preceding claims, wherein the flexion and extension movement comprises at least one phase wherein the rotation and the translation of the second support relative to the first support occurs simultaneously.

19. The apparatus of any one of the preceding claims, wherein the flexion and extension movement comprises at least one phase wherein the rotation of the second support relative to the first support occurs substantially without the translation of the second support relative to the first support.

20. The apparatus of claim any one of the preceding claims, wherein during a full flexion movement of the second support relative to the first support from a maximum degree of extension to a maximum degree of flexion, a highest rate of translation of the second support relative to the first support occurs during an intermediate phase of the full flexion movement.

21. The apparatus of claim 20, wherein the intermediate phase is between 20% and 60%, or 30% and 50%, of the full flexion movement.

22. The apparatus of any one of the preceding claims, wherein during a full flexion movement of the second support relative to the first support from a maximum degree of extension to a maximum degree of flexion, a lowest rate of translation, or substantially no translation, of the second support relative to the first support, occurs during a beginning and/or end phase of the full flexion movement.

23. The apparatus of claim 22, wherein the beginning phase is between 0% and 30%, or 0% and 20% of the full flexion movement.

24. The apparatus of claim 22 or 23, wherein the end phase is between 50% and 100%, or 60% and 100% of the full flexion movement.

25. The apparatus of any one of the preceding claims, wherein the mechanical system comprises first and second arms configured to be fixed to the first support and the second support, respectively, and to extend at least partially along a length of the first support and the second support, respectively.

26. The apparatus of claim 25, wherein the mechanical system is configured to relatively rotate and/or translate the first and second arms to cause the relative rotation and translation of the first and second supports.

27. The apparatus of claim 25 or claim 26, wherein a length of the first and/or second arm is adjustable.

28. The apparatus of claim 27, wherein the first and/or second arm comprises first and second arm portions that are slidable relative to each other to adjust the length the first and/or second arm.

29. The apparatus of any one of the preceding claims, further comprising an extension limiter configured to limit a maximum degree of extension of the second support relative to the first support.

30. The apparatus of claim 29, wherein the extension limiter is adjustable to change the maximum degree of extension of the second support relative to the first support.

31. The apparatus of any one of the preceding claims, comprising a flexion limiter configured to limit a maximum degree of flexion of the second support relative to the first support.

32. The apparatus of claim 31, wherein the flexion limiter is adjustable to change the maximum degree of flexion of the second support relative to the first support.

33. The apparatus of any one of the preceding claims, further comprising one or more indicators configured to indicate a degree of flexion of the second support relative to the first support achieved during the flexion movement and/or a degree of extension of the second support relative to the first support achieved during the extension movement.

34. The apparatus of claim 33, wherein each indicator comprises a selection element, and one or more indicia elements movable relative to the selection element during the flexion and/or extension movement.

35. The apparatus of claim 33 or 34, wherein the one or more indicators are positioned to be directly observable by the wearer of the apparatus.

36. The apparatus of claim 33, 34 or 35, wherein the one or more indicators are positioned on or at a peripheral surface of a housing of the mechanical system.

37. The apparatus of any one of the preceding claims, wherein one or both of the first support and the second support comprise one or more straps configured to be secured to the respective first limb portion or second limb portion.

38. The apparatus of any one of the preceding claims, wherein one or both of the first support and the second support comprise one or more sleeve portions configured to be secured to the respective first limb portion or second limb portion.

39. The apparatus of any one of the preceding claims, wherein one or both of the first support and the second support are secured to the respective first limb portion or second limb portion using one or more fasteners.

40. The apparatus of claim 39, wherein the one or more fasteners comprise one or more of:
a hook and loop fastener;
a buckle fastener;
a snap fastener; and
a hook-eye fastener.

41. The apparatus of any one of the preceding claims, wherein the first support and the second support comprise one or more pads.

42. The apparatus of any one of the preceding claims, wherein the joint is a knee joint, an elbow joint or an ankle joint.

43. The apparatus of any one of the preceding claims, wherein the apparatus is a knee brace, an elbow brace or an ankle brace.

44. A method of performing joint range of motion rehabilitation comprising:
securing a first support to a first limb portion of a wearer at a first side of a joint;
securing a second support to a second limb portion of the wearer at a second side of the joint; and
operating a mechanical system connected to the first support and the second support to control flexion and extension movement of the second support relative to the first support, wherein during the flexion and extension movement, the second support rotates and translates relative to the first support.

45. The apparatus of claim 44, wherein operation of the mechanical system comprises using an actuator to partially or entirely drive the flexion and extension movement of the second support relative to the first support.

46. The apparatus of claim 45, wherein the actuator is operable by the wearer.

47. The apparatus of claim 44, 45 or 46, wherein operation of the mechanical system comprises the wearer partially or entirely driving the flexion and extension movement of the second support relative to the first support by carrying out native flexion and extension movement of the second limb portion relative to the first limb portion.
